(19) United States
(12) Patent Application Publication
(10) Pub. No.: US 2026/0345539 A1
(43) Pub. Date: Nov. 5, 2026

(54) VALINE AND ISOLEUCINE FORMULATIONS

(71) Applicant: Meta Healthcare Ltd, Gloucestershire (GB)

(72) Inventors: Stephen Tickle, Frodsham (GB); Christopher Wagstaff, Cambridge (GB)

(73) Assignee: Meta Healthcare Ltd, Gloucestershire (GB)

(21) Appl. No.: 19/285,866

(22) Filed: Jul. 30, 2025

(30) Foreign Application Priority Data
Apr. 30, 2025 (GB) 2506599.6

(51) Int. Cl. A61K 31/198; A61K 47/02; A61K 47/12; A61K 47/38

(52) U.S. Cl. CPC A61K 31/198; A61K 47/02; A61K 47/12; A61K 47/38

(57) ABSTRACT

This disclosure relates to a liquid formulation of valine and isoleucine. The liquid formulations of valine and isoleucine are also relevant for use in methods of treatment, such as maple syrup urine disease.

### CROSS-REFERENCE TO RELATED APPLICATION

This application claims priority to Great Britain Patent Application No. 2506599.6, filed 30 Apr. 2025, entitled VALINE AND ISOLEUCINE FORMULATIONS, the disclosure of which is hereby incorporated herein by reference.

### TECHNICAL FIELD

This disclosure relates to a liquid formulation that includes valine or isoleucine. The liquid formulations of valine or isoleucine are also relevant for use in methods of treatment. The liquid formulations are also contemplated for use in the treatment of maple syrup urine disease.

### BACKGROUND

Valine and isoleucine are naturally occurring amino acids essential for human life. They are consequently very important for the maintenance of human health. Both valine and isoleucine can be consumed as a dietary supplement, nutritional supplement, exercise supplement, or health supplement. These supplements take on the form of solid powders.

The solid powders are consumed in tablet form or in combination with food or drink. Combination with food or drink generally leads to undesirable variability of administration. The full dose may not be consumed or the full dose may not be appropriately metabolised. In particular, complete dissolution within a drink can be problematic. This is particularly true of valine and isoleucine which both have challenging dissolution profiles. Neither solid is particularly soluble in water, with valine having a complete dissolution time in the order of hours.

Existing powder formulations of valine and isoleucine also include carbohydrate. As will be readily understood, excessive processed carbohydrate consumption must be carefully controlled. Therefore, it is desirable to have a method of administration that avoids contemporaneous administration of superfluous carbohydrate. Powder formulations also often contain additives to improve characteristics of the powder, such as flowability and stability. These additives often contain sodium salts or other salts which can increase the amount of salts, such as sodium, in a patients diet. This can have long term negative effects on the patient.

Valine and isoleucine supplements are used in a variety of ways. One example is in the treatment of maple syrup urine disease (MSUD). Treatment of MSUD consists of natural protein restriction, limiting intake of leucine (Leu), valine (Val), and isoleucine (Ile), together with other natural amino acids. This is to support anabolism. Although restriction of dietary amino acids is necessary to treat MSUD, additional Val and Ile supplements are commonly required to further promote anabolism and prevent Val and Ile deficiency. Anabolism accelerates blood Leu reduction. In MSUD, a lifelong dietary regimen with restricted Leu intake is essential to prevent neurotoxicity, which if uncontrolled can lead to encephalopathy. Plasma branched-chain amino acids (BCAA) are maintained within a narrow target treatment range. Dietary restriction of Leu, Val and Ile is necessary, whilst avoiding their deficiency. The diet is supplemented with BCAA-free amino acids.

Therefore, it is important that blood levels of Val and Ile are at an appropriate biological level to avoid a deficiency and also an appropriate level to promote anabolism. Utilising powdered Val and Ile results in increased blood concentrations but with a high degree of variance, introduced by the variance of dissolution and amount administered by admixture with drinks or food.

In Europe, including the United Kingdom (UK), three companies provide single Val and Ile supplements. In the UK, powdered supplements of valine Val and Ile (50 mg) are provided in pre-measured sachets (Valine 50, Isoleucine 50, Vitaflo International) and are available on Advisory Committee on Borderline Substances (ACBS) prescription. Carbohydrate (CHO) (3.8 g per 4 g sachet) is added to each 50 mg Val or Iso sachet. This CHO must be considered and calculated in infant and emergency feeds. These powders require reconstitution with water or can be mixed with drinks such as squash or low protein milk but affect their taste. They may be added to powdered protein substitutes but are impractical with liquid protein substitutes. Nutricia also produce 100 g tubs of Val and Ile supplements. These supplements are not practical and not widely used. They contain 100 g of Val or Ile per 100 g tub and require very careful measurement as only small quantities are required. Another company (MetaX) produces single amino acids of Val and Ile in tablet or powder form, but these products are not available in the UK.

Although the premeasured sachets of powder were convenient, requiring no measuring, they do not mix well in water or powder, some may be left behind in the sachets as the sachets were not transparent. This led to the possibility to underdose.

### SUMMARY

The present disclosure aims to provide a liquid formulation. The liquid formulation provides a known amount of valine or isoleucine for oral administration. This improves on the known products for administering valine and isoleucine which struggle with variability and under dosing.

In certain embodiments, the present disclosure aims to provide a formulation with reduced salt content compared to an equivalent dose of a prior art composition.

In certain embodiments, the present disclosure aims to provide an improved dosing reliability. In certain embodiments, the present disclosure aims to reduce the daily dose of administered valine and isoleucine.

The present disclosure also aims to avoid the need for prolonged dietetic calculation. Although the dosing of powder supplements has a high degree of variance, as discussed above, it is essential that the amount of Val and Ile that is administered is as close as possible to the required amount to provide the appropriate blood levels. Thus, calculations are required to determine how much powder must be mixed to provide the appropriate amount to the patient. This must take into account the concentration of Val or Ile in the powder amongst other factors. The formulation of the present disclosure aims to reduce the burden of dietetic calculation.

In certain embodiments of the present disclosure, the formulation aims to improve convenience for patients. In certain embodiments of the present disclosure, the formulation aims to be easier to administer. In certain embodiments of the present disclosure, the formulation aims to be palatable and well tolerated. In certain embodiments of the present disclosure, the formulation aims to improve patient adherence to a treatment regimen.

In accordance with the present disclosure there is provided a liquid formulation comprising valine or isoleucine. In certain embodiments the liquid formulation is suitable for oral administration. Therefore, in certain embodiments, there is provided an oral liquid formulation comprising valine or isoleucine.

In certain embodiments the valine or isoleucine is present in an amount of from about 1.0 w/v % to about 10 w/v %. In certain embodiments the valine or isoleucine is present in an amount of from about 1.0 w/v % to about 7.5 w/v % or about 2.0 w/v % to about 6.0 w/v %. In certain embodiments valine is present in an amount of about 5.0 w/v %. In certain embodiments isoleucine is present in an amount of about 2.5 w/v %.

In certain embodiments, the liquid formulation further comprises: a buffer, a preservative, a sweetener, a flavouring, a viscosity modifier or a combination thereof.

In certain embodiments the buffer is selected from: citric acid, trisodium citrate, disodium hydrogen orthophosphate, disodium hydrogen phosphate, sodium dihydrogen phosphate or any combination thereof. The buffer system may be formed of solvates or hydrates. For example, the buffer may be selected from: citric acid, trisodium citrate dihydrate, disodium hydrogen orthophosphate dihydrate, disodium hydrogen phosphate dihydrate, sodium dihydrogen phosphate dihydrate or any combination thereof.

The buffer may be present as a pair, representative pairs are: citric acid and disodium hydrogen orthophosphate; citric acid and trisodium citrate; or disodium hydrogen phosphate and sodium dihydrogen phosphate.

In certain embodiments the buffer is selected from citric acid and disodium hydrogen orthophosphate. The buffer system may be formed of solvates or hydrates. For example, the buffer may be citric acid anhydrous and disodium hydrogen orthophosphate dihydrate.

In certain embodiments, the formulation comprises a buffer in a concentration of from about 0.5 to about 5% w/v. In certain embodiments, the formulation comprises a preservative in a concentration of from about 0.5 to about 4% w/v. In certain embodiments, the formulation comprises a preservative in a concentration selected from about 0.8% w/v, about 2.1% w/v, and about 2.9% w/v.

In certain embodiments, the formulation comprises a buffer in a concentration of from about 0.1 to about 2% w/v. In certain embodiments, the formulation comprises a buffer in a concentration of from 0.2 to 1.5% w/v. In certain embodiments, the formulation comprises a buffer in a concentration selected from about 0.25% w/v, about 0.72% w/v, and about 0.97% w/v.

In certain embodiments the preservative is selected from methyl para hydroxybenzoate, ethyl para hydroxybenzoate, propyl para hydroxybenzoate, butyl para hydroxybenzoate, salts thereof or a combination thereof. In certain embodiments the preservative is selected from sodium methyl para hydroxybenzoate and sodium propyl para hydroxybenzoate, or a combination thereof.

In certain embodiments, the formulation comprises a preservative in a concentration of from about 0.05 to about 0.5% w/v. In certain embodiments, the formulation comprises a preservative in a concentration of from about 0.1 to about 0.4% w/v. In certain embodiments, the formulation comprises a preservative in a concentration selected from about 0.2% w/v, about 0.22% w/v, about 0.25% w/v and about 0.3% w/v.

In certain embodiments the ratio of sodium methyl para hydroxybenzoate to sodium propyl para hydroxybenzoate is from 20:1 to 2:1 or 15:1 to 5:1. In certain embodiments the ratio of sodium methyl para hydroxybenzoate to sodium propyl para hydroxybenzoate is 10:1.

In certain embodiments the preservative is sodium methyl parabens in an amount of about 0.2% w/v and sodium propyl parabens about 0.02% w/v.

In certain embodiments, the formulation may be at a pH in the range of from 2 to 12. Optionally, the pH is from 4 to 10 or the pH is from 5 to 7. In certain embodiments, the formulation may have a pH of 5.5, 6.0, or 6.5.

The sweetener may be selected from: sucralose, potassium acesulfame, aspartame sodium saccharin and combinations thereof. The sweetener may be selected from sodium saccharin.

The sweetener may be a solvate or hydrate. For example, sodium saccharin monohydrate or sodium saccharin dihydrate.

In certain embodiments, the formulation comprises a sweetener in a concentration of from 0 to about 0.5% w/v. In certain embodiments, the formulation comprises a sweetener in a concentration of from about 0.1 to about 0.4% w/v. In certain embodiments, the formulation comprises a sweetener in a concentration of 0, about 0.1% w/v, about 0.15% w/v, about 0.2% w/v and about 0.4% w/v.

In certain embodiments, the formulation comprises a flavouring in a concentration of from 0 to 0.5% w/v. In certain embodiments, the formulation comprises a sweetener in a concentration of from 0.1 to 0.5% w/v. In certain embodiments, the formulation comprises a sweetener in a concentration of 0, 0.15% w/v, 0.25% w/v and 0.5% w/v.

The flavouring may be strawberry flavour (PHS-180152).

In certain embodiments, the viscosity modifier is selected from hydroxyethylcellulose.

The liquid formulation further comprises a pharmaceutically acceptable liquid carrier. The liquid carrier is capable of dissolving valine and isoleucine. Preferably, the pharmaceutically acceptable carrier is water. Therefore, the liquid formulation of the present disclosure may be an aqueous liquid formulation.

In certain embodiments, the formulation comprises a viscosity modifier in a concentration of from 0 to about 1.0% w/v. In certain embodiments, the formulation comprises a sweetener in a concentration of from about 0.3 to about 0.7% w/v or about 0.3 to about 0.4% w/v. In certain embodiments, the formulation comprises a sweetener in a concentration of 0, about 0.1% w/v, about 0.15% w/v, about 0.2% w/v and about 0.4% w/v.

In certain embodiments, the formulation comprises valine or isoleucine, a preservative, a buffer, and water. In certain embodiments, the formulation comprises valine, a preservative, a buffer, a sweetener, a flavouring, a viscosity modifier and water.

In certain embodiments, the formulation comprises isoleucine, a preservative, a buffer, and water. In certain embodiments, the formulation comprises isoleucine, a preservative, a buffer, a sweetener, a flavouring, a viscosity modifier and water.

In certain embodiments, the formulation comprises valine, sodium methyl parabens, sodium propyl parabens, sodium saccharin, citric acid anhydrous, disodium hydrogen orthophosphate dihydrate, hydroxyethylcellulose, strawberry flavour, and purified water.

In certain embodiments, the formulation comprises isoleucine, sodium methyl parabens, sodium propyl parabens, sodium saccharin, citric acid anhydrous, disodium hydrogen orthophosphate dihydrate, hydroxyethylcellulose, strawberry flavour, and purified water.

In an embodiment, the formulation is:

| | Concentration |
|---|---|
| Raw Materials | % w/v |
| Valine | 5.0 |
| Sodium Methyl Parabens | 0.206 |
| Sodium Propyl Parabens | 0.0224 |
| Sodium Saccharin | 0.15 |
| Citric Acid Anhydrous | 0.76 |
| Disodium Hydrogen Orthophosphate Dihydrate | 2.153 |
| Natrosol 250HX | 0.4 |
| Strawberry Flavour (PHS-180152) | 0.5 |
| Purified Water Ph. Eur. | To 100 |

In an embodiment, the formulation is:

| | Concentration |
|---|---|
| Raw Materials | % w/v |
| Isoleucine | 2.5 |
| Sodium Methyl Parabens | 0.206 |
| Sodium Propyl Parabens | 0.0224 |
| Sodium Saccharin | 0.15 |
| Citric Acid Anhydrous | 0.76 |
| Disodium Hydrogen Orthophosphate Dihydrate | 2.153 |
| Natrosol 250HX | 0.4 |
| Strawberry Flavour (PHS-180152) | 0.5 |
| Purified Water Ph. Eur. | To 100 |

In an embodiment, the formulation is:

| | Concentration |
|---|---|
| Raw Materials | % w/v |
| Valine | 5.0 |
| Sodium Methyl Parabens | 0.206 |
| Sodium Propyl Parabens | 0.0224 |
| Sodium Saccharin | 0.15 |
| Citric Acid Anhydrous | 0.25 |
| Disodium Hydrogen Orthophosphate Dihydrate | 0.72 |
| Natrosol 250HX | 0.4 |
| Strawberry Flavour (PHS-180152) | 0.5 |
| Purified Water Ph. Eur. | To 100 |

In an embodiment, the formulation is:

| | Concentration |
|---|---|
| Raw Materials | % w/v |
| Isoleucine | 2.5 |
| Sodium Methyl Parabens | 0.206 |
| Sodium Propyl Parabens | 0.0224 |
| Sodium Saccharin | 0.15 |
| Citric Acid Anhydrous | 0.25 |
| Disodium Hydrogen Orthophosphate Dihydrate | 0.72 |
| Natrosol 250HX | 0.4 |
| Strawberry Flavour (PHS-180152) | 0.5 |
| Purified Water Ph. Eur. | To 100 |

In an aspect of the present disclosure there is provided a formulation of the present disclosure for use in a method of treatment.

In an aspect of the present disclosure the formulation of the present disclosure is for use in a method of treating maple syrup urine disease or urea cycle disorders.

In an aspect of the disclosure there is provided a method of treatment, where in the method comprises administering a therapeutic amount of the formulation of the present disclosure.

In an aspect of the disclosure there is provided a method of treatment of maple syrup urine disease or urea cycle disorders, where in the method comprises administering a therapeutic amount of the formulation of the present disclosure.

Administration of the formulation may be oral.

In an aspect of the disclosure there is provided a kit comprising the formulation of the disclosure comprising valine and a formulation of the disclosure comprising isoleucine.

### DETAILED DESCRIPTION

Concentrations within this application have been provided as percentage of weight by volume (% w/v). This concentration is a measure of mass per volume of total liquid volume of the formulation, put another way the concentration can be measured as g/L. For example, 50 g of a component in a liquid of total volume of 1 litre would give a % w/v of 5% w/v.

Where a buffer is present, in any embodiment discussed herein, it is contemplated that the buffer may be a two-component buffer system. Thus, any disclosure of the concentration of buffer may refer to one of the two buffers or the total concentration of both buffers in the buffer system.

Where a preservative is present, in any embodiment discussed herein, it is contemplated that the preservative may be a two-component preservative system. Thus, any disclosure of the concentration of preservative may refer to one of the two preservatives or the total concentration of both preservatives in the preservative system.

### EXAMPLES

### Example 1

A formulation of the present disclosure was prepared following the below process. This method is suitable for the manufacture of Valine 50 mg/ml Oral Solution.
1) Into the mixing vessel add 75% of the total volume of water required.
2) Add the sodium methyl and propyl parabens and stir to dissolve.
3) Add the Disodium Hydrogen Orthophosphate and stir to dissolve.
4) Add the Citric acid and stir to dissolve.
5) Add the sodium saccharin and stir to dissolve.
6) Add the strawberry flavour and stir to dissolve.
7) Add the Valine and stir to dissolve.
8) Slowly sprinkle in the hydroxyethylcellulose (Natrosol 250HX) with stirring until fully wetted and dissolved.
9) Dilute to volume with water and
10) Fill 100 ml into appropriate size amber glass bottles and apply a tamper-evidence/child-resistant (TE/CR) Closure.

Steps 3 and 4 may be interchangeable.

An example valine formulation is shown below.

| | Concentration |
|---|---|
| Raw Materials | % w/v |
| Valine | 5.0 |
| Sodium Methyl Parabens | 0.206 |
| Sodium Propyl Parabens | 0.0224 |
| Sodium Saccharin | 0.15 |
| Citric Acid Anhydrous | 0.76 |
| Disodium Hydrogen Orthophosphate Dihydrate | 2.153 |
| Natrosol 250HX | 0.4 |
| Strawberry Flavour (PHS-180152) | 0.5 |
| Purified Water Ph. Eur. | To 100 |

*Natrosol 250HX is a viscosity modifier - hydroxyethylcellulose.

An amber 100 ml glass type III (kylix syrup) bottle with a Pilfer-Proof (PP) 28 mm neck was sourced from Adelphi packaging was used. The bottles are manufactured by SGD Pharma in France. A suitable cap was also sourced from Adelphi packaging a 28 mm White Tamper Evident & Child Res Screw Cap TriSeal Wad. The caps were manufactured by Stella GmbH in Germany.

### Example 2

An isoleucine formulation can be prepared in the same manner as set out in Example 1 substituting the valine for isoleucine to provide a formulation as set out below.

| | Concentration |
|---|---|
| Raw Materials | % w/v |
| Isoleucine | 2.5 |
| Sodium Methyl Parabens | 0.206 |
| Sodium Propyl Parabens | 0.0224 |
| Sodium Saccharin | 0.15 |
| Citric Acid Anhydrous | 0.76 |
| Disodium Hydrogen Orthophosphate Dihydrate | 2.153 |
| Natrosol 250HX | 0.4 |
| Strawberry Flavour (PHS-180152) | 0.5 |
| Purified Water Ph. Eur. | To 100 |

### Example 3

A laboratory scale batch of Valine 50 mg/ml Oral Solution, according to Example 1, was prepared and filled into 100 ml amber glass bottles and capped with tamper evident child resistant closures, as set out above. The samples were placed on storage at 25 °C/60% RH. The test results at 0 months, 3 months and 6 months are set out below in Table 1a and 1b.

TABLE 1a

| Test | Specification | 0 | 3 | 6 |
|---|---|---|---|---|
| Appearance | Clear slightly viscous straw-coloured | Complies | Complies | Complies |
| | liquid with odour of strawberry | | | |
| Identification | The retention time of the principal | Complies | Complies | Complies |
| | peak in the assay sample | | | |
| | chromatogram corresponds with the | | | |
| | retention time of the Valine peak in | | | |
| | the assay standard chromatogram | | | |
| pH | 5.5-6.5 | 6.28 | 6.34 | 6.29 |
| Weight per | To be recorded | 1.0321 g/ml | 1.0318 g/ml | 1.0319 g/ml |
| ml | | | | |
| Volume of | Not less than 100 ml | 101.4 ml | | |
| Fill | | | | |

TABLE 1b

| Test | Specification | 0 | 3 | 6 |
|---|---|---|---|---|
| Parabens Assay | Sodium Methyl Parabens | 0.204% | 0.200% | 0.200% |
| | 0.0185-0.0227% w/v | w/v | w/v | w/v |
| | Sodium Propyl Parabens | 0.0222% | 0.0214% | 0.0214% |
| | 0.0202-0.0246% w/v | w/v | w/v | w/v |
| Valine Assay | 47.5-52.5 mg/ml | 50.6 | 50.3 | 50.8 |
| | | mg/ml | mg/ml | mg/ml |
| Related | Isoleucine NMT 0.4% wrta | Non- | Non- | Non- |
| Substances | Individual unknown impurities | Detected | Detected | Detected |
| | NMT 0.1% wrta | | | |
| | Total Impurities NMT 1.0% | | | |
| | wrta | | | |
| Microbial | NMT 10² TAMC (CFU/ml) | <10 | | <10 |
| | NMT 10¹ TYMC (CFU/ml) | <10 | | <10 |
| | Absence of E coli in 1 ml | Not | | Not |
| | | Detected | | Detected |
| | | 117YE07 | | 130YE11A |
| Preservative | Meets Criteria for Oral Liquids | Complies | | |
| Efficacy | | 33E19 | | |

The results show that the liquid oral formulation of the present disclosure meets all stability requirements over 6 months at 25 °C/60% RH.

### Example 4

A laboratory scale batch of Valine 50 mg/ml Oral Solution was prepared, according to Example 1, and filled into 100 ml amber glass bottles and capped with tamper evident child resistant closures, as set out above. The samples were placed on storage at 40 °C/70% RH. The test results at 0 months, 3 months and 6 months are set out below in Table 2a and 2b.

TABLE 2a

| Test | Specification | 0 | 3 | 6 |
|---|---|---|---|---|
| Appearance | Clear slightly viscous straw- | Complies | Complies | Clear orange |
| | coloured liquid with odour of | | | coloured |
| | strawberry | | | solution with |
| | | | | odour of |
| | | | | strawberry |
| Identification | The retention time of the | Complies | Complies | Complies |
| | principal peak in the assay | | | |
| | sample chromatogram | | | |
| | corresponds with the retention | | | |
| | time of the Valine peak in the | | | |
| | assay standard chromatogram | | | |
| pH | 5.5-6.5 | 6.28 | 6.35 | 6.26 |
| Weight per | To be recorded | 1.0321 g/ml | 1.0324 g/ml | 1.0321 g/ml |
| ml | | | | |
| Volume of | Not less than 100 ml | 101.4 ml | | |
| Fill | | | | |

TABLE 2b

| Test | Specification | 0 | 3 | 6 |
|---|---|---|---|---|
| Parabens Assay | Sodium Methyl Parabens | 0.204% | 0.197% | 0.191% |
| | 0.0185-0.0227% w/v | w/v | w/v | w/v |
| | Sodium Propyl Parabens | 0.0222% | 0.0212% | 0.0209% |
| | 0.0202-0.0246% w/v | w/v | w/v | w/v |
| Valine Assay | 47.5-52.5 mg/ml | 50.6 | 50.0 | 50.5 |
| | | mg/ml | mg/ml | mg/ml |
| Related | Isoleucine NMT 0.4% wrta | Non- | Non- | Non- |
| Substances | Individual unknown impurities | Detected | Detected | Detected |
| | NMT 0.1% wrta | | | |
| | Total Impurities NMT 1.0% | | | |
| | wrta | | | |
| Microbial | NMT 10² TAMC (CFU/ml) | <10 | | <10 |
| | NMT 10¹ TYMC (CFU/ml) | <10 | | <10 |
| | Absence of E coli in 1 ml | Not | | Not |
| | | Detected | | Detected |
| | | 117YE07 | | 130YE11B |
| Preservative | Meets Criteria for Oral Liquids | Complies | | |
| Efficacy | | 33E19 | | |

The results show that the liquid oral formulation of the present disclosure meets all stability requirements over 6 months at 40 °C/75% RH.

### Example 5

A laboratory scale batch of isoleucine 25 mg/ml Oral Solution was prepared, as per Example 2, and filled into 100 ml amber glass bottles and capped with tamper evident child resistant closures, as set out above. The samples were placed on storage at 25 °C/60% RH. The test results at 0 months, 3 months and 6 months are set out in Table 3a and 3b.

TABLE 3a

| Test | Specification | 0 | 3 | 6 |
|---|---|---|---|---|
| Appearance | Clear slightly viscous straw-coloured | Complies | Complies | Complies |
| | liquid with odour of strawberry | | | |
| Identification | The retention time of the principal | Complies | Complies | Complies |
| | peak in the assay sample | | | |
| | chromatogram corresponds with the | | | |
| | retention time of the Isoleucine | | | |
| | peak in the assay standard | | | |
| | chromatogram | | | |
| pH | 5.5-6.5 | 6.15 | 6.18 | 6.18 |
| Weight per | To be recorded | 1.0260 g/ml | 1.0262 g/ml | 1.0257 g/ml |
| ml | | | | |
| Volume of | Not less than 100 ml | 101.3 ml | | |
| Fill | | | | |

TABLE 3b

| Test | Specification | 0 | 3 | 6 |
|---|---|---|---|---|
| Parabens Assay | Sodium Methyl Parabens | 0.203% | 0.201% | 0.200% |
| | 0.0185-0.0227% w/v | w/v | w/v | w/v |
| | Sodium Propyl Parabens | 0.0225% | 0.0220% | 0.0215% |
| | 0.0202-0.0246% w/v | w/v | w/v | w/v |
| Isoleucine Assay | 23.75-26.25 mg/ml | 25.0 | 25.65 | 25.46 |
| | | mg/ml | mg/ml | mg/ml |
| Related | Valine NMT 0.3% wrta | Non- | Non- | Non- |
| Substances | Individual unknown impurities | Detected | Detected | Detected |
| | NMT 0.1% wrta | | | |
| | Total Impurities NMT 1.0% wrta | | | |
| Microbial | NMT 10² TAMC (CFU/ml) | <10 | | <10 |
| | NMT 10¹ TYMC (CFU/ml) | <10 | | <10 |
| | Absence of E coli in 1 ml | Not | | Not |
| | | Detected | | Detected |
| | | 117YE08 | | 130YE10A |
| Preservative | Meets Criteria for Oral Liquids | Complies | | |
| Efficacy | | 33E20 | | |

The results show that the liquid oral formulation of the present disclosure meets all stability requirements over 6 months at 25 °C/60% RH.

### Example 6

A laboratory scale batch of isoleucine 25 mg/ml Oral Solution was prepared, as per Example 2, and filled into 100 ml amber glass bottles and capped with tamper evident child resistant closures, as set out above. The samples were placed on storage at 40 °C/70% RH. The test results at 0 months, 3 months and 6 months are set out below in Table 4a and 4b.

TABLE 4a

| Test | Specification | 0 | 3 | 6 |
|---|---|---|---|---|
| Appearance | Clear slightly viscous straw- | Complies | Complies | Clear |
| | coloured liquid with odour of | | | yellow/orange |
| | strawberry | | | coloured solution |
| | | | | with odour of |
| | | | | strawberry |
| Identification | The retention time of the | Complies | Complies | Complies |
| | principal peak in the assay | | | |
| | sample chromatogram | | | |
| | corresponds with the | | | |
| | retention time of the | | | |
| | Isoleucine peak in the assay | | | |
| | standard chromatogram | | | |
| pH | 5.5-6.5 | 6.15 | 6.23 | 6.17 |
| Weight per | To be recorded | 1.0260 g/ml | 1.0269 g/ml | 1.0255 g/ml |
| ml | | | | |
| Volume of | Not less than 100 ml | 101.3 ml | | |
| Fill | | | | |

TABLE 4b

| Test | Specification | 0 | 3 | 6 |
|---|---|---|---|---|
| Parabens Assay | Sodium Methyl Parabens | 0.203% | 0.197% | 0.193% |
| | 0.0185-0.0227% w/v | w/v | w/v | w/v |
| | Sodium Propyl Parabens | 0.0225% | 0.0213% | 0.0210% |
| | 0.0202-0.0246% w/v | w/v | w/v | w/v |
| Isoleucine Assay | 23.75-26.25 mg/ml | 25.0 | 25.75 | 25.61 |
| | | mg/ml | mg/ml | mg/ml |
| Related | Valine NMT 0.3% wrta | Non- | Non- | Non- |
| Substances | Individual unknown impurities | Detected | Detected | Detected |
| | NMT 0.1% wrta | | | |
| | Total Impurities NMT 1.0% wrta | | | |
| Microbial | NMT 10² TAMC (CFU/ml) | <10 | | <10 |
| | NMT 10¹ TYMC (CFU/ml) | <10 | | <10 |
| | Absence of E coli in 1 ml | Not | | Not |
| | | Detected | | Detected |
| | | 117YE08 | | 130YE10B |
| Preservative | Meets Criteria for Oral Liquids | Complies | | |
| Efficacy | | 33E20 | | |

The results show that the liquid oral formulation of the present disclosure meets all stability requirements over 6 months at 40 °C/75% RH.

### Example 7

The valine composition of Example 1 and the isoleucine composition of Example 2 were included in an efficacy trial. The efficacy trial provided positive results demonstrating that the liquid compositions of the present disclosure enabled a reduction in the dose of valine required to obtain a therapeutically effective amount. The therapeutic amount was judged by comparing blood levels of valine and isoleucine to target blood levels. Target blood levels were set based on the amount of powder administered to the patient.

Study design: The study was an observational, prospective, open-label observational study conducted over a 56-day period in five paediatric patients with MSUD (2 boys, 3 girls). The study was structured in two phases:

Phase 1 (Day 0-28): Patients received the liquid Val formulation (same dosage as their usual powdered supplement) while continuing the use of powdered Ile.

Phase 2 (Day 29-56): Patients returned to powdered Val and substituted the powdered Ile with the new liquid formulation.

Subject inclusion criteria: male or female participants, aged ≥1 to 16 years, with a confirmed diagnosis of classical MSUD, already prescribed Val and Ile supplementation as part of their dietary management. They had to be in a stable clinical condition. Participants and their caregivers needed to be able to understand and comply with the study procedures and provide written informed consent/assent. Children were excluded if they had undergone liver transplantation or had any comorbidity or acute illness that could interfere with study outcomes. Additional exclusion criteria included a history of poor adherence to treatment.

Clinical assessment: Data was collected at three time points: study baseline (T0), Day 28 (T1), and Day 56 (T2). During each visit, general health status was assessed through caregiver interviews, collecting data on overall health, any illness, symptoms or adverse reactions potentially related to the RTU formulations. Information, via a daily questionnaire was collected about adherence and gastrointestinal (GI) tolerance, collecting data on any GI symptoms e.g., nausea, bloating, flatulence, vomiting.

Biochemical assessment: Weekly plasma concentrations of Val, Ile and Leu were collected by the parents at home, sent to the hospital reference laboratory and analysed to monitor metabolic control and response to the new RTU formulations during the intervention. In the month preceding the study, weekly blood samples were collected to assess metabolic stability.

Nutritional assessment: At each time point (T0, T1, and T2), the following were collected: 3-day food diary recording all food/drink intake including protein substitute, and anthropometry (weight and height).

Daily adherence with Val and Ile supplement administration were reported. Daily ease of use, issues with administration and any gastro-intestinal side effects were documented by the caregivers.

### Product Acceptability and Tolerance

At the beginning and end of each intervention phase, participants completed a structured qualitative questionnaire with caregivers comparing their usual powdered Val and Ile supplements and the study Val and Ile. Key aspects evaluated included adherence, smell, taste, palatability, aftertaste and ease of use of the Val and Ile supplements. Response options included: Great, Good, OK, Bad, Terrible, and N/A. For analysis purposes, responses were converted to numerical values as follows: Great=5, Good=4, OK=3, Bad=2, and Terrible=1.

The study was conducted in adherence to the guidelines of the Local Ethical Committee and performed in accordance with the ethical standards of the 2024 Helsinki Declaration. Informed consent for the treatment of anonymized data was obtained from a parent or a legal guardian.

### Participants

Five patients with classical MSUD, three females and two males, median age 8.8 years (range 3 to 14 years) were recruited. Their ethnicity was British Pakistani (n=4) and British Bangladeshi (n=1). One was identified by newborn screening, n=2 diagnosed by clinical symptoms in the neonatal period, and n=2 by sibling screening.

### Gastrointestinal Tolerance

Liquid Val and Ile was well tolerated. For the majority of patients, mild symptoms were reported at T0, which either improved or remained stable at T1 and T2.

### Biochemical Assessment

Routine weekly blood samples were sent to the reference laboratory at Birmingham Children's Hospital. Table 5 reports the mean (range) blood levels for the study period. Pre-study plasma BCAA were: Val 426 μmol/L (range: 289-516), Ile 300 μmol/L (range: 243-312) and Leu 356 μmol/L (range: 214-716). Leu levels were highest at baseline but decreased with liquid Val supplementation (Leu mean 253 μmol/L, range 195-349) and liquid Ile (Leu mean 234 μmol/L, range 106-324).

Plasma Ile remained stable from the study start to end, start mean Ile 300 μmol/L (range 243-312) and study end 327 μmol/L (239-366). Plasma Val levels were elevated at baseline (mean 426 μmol/L, range 289-516) and remained stable during liquid Val supplementation (mean 424 μmol/L, range 301-513), and showed a slight increase with liquid Ile (mean 433 μmol/L, range 324-610). Target plasma levels were: 100-300 μmol/L for Leu, 200-400 μmol/L for Val, and 200-400 μmol/L for Ile.

However, the required dose of liquid Val and Ile decreased over the 4-week period. At the study baseline, the amount of Val supplementation was a mean of 150 mg/day (range 100-200), and Ile 200 mg/day (range 150-300). At the end of the study, the Ready-to-Use (RTU) supplements decreased by 30% for Val (median dose, 100 mg/day, range 50-150) and 18.4% for Ile (median dose, 125 mg/day, range 100-300) compared to powdered supplements (Table 6).

Table 5. Means and range (minimum-maximum) of weekly Val, Ile and Leu levels. Data presented is taken pre-study, before T0; on liquid Val, from T0 to T1; and on liquid Ile, from T1 to T2.

TABLE 5

| | | Mean blood results and range (μmol/L) | | |
|---|---|---|---|---|
| Patients | Time of collection | Leucine | Isoleucine | Valine |
| N.1 | Pre-study | 254 (185-370) | 243 (212-266) | 442 (412-480) |
| | On liquid Valine | 251 (129-351) | 251 (171-309) | 513 (361-703) |
| | On liquid Isoleucine | 238 (223-264) | 266 (220-312) | 487 (428-594) |
| N.2 | Pre-study | 716 (167-1278)* | 381 (262-485) | 516 (428-594) |
| | On liquid Valine | 199 (113-378) | 284 (248-319) | 443 (291-555) |
| | On liquid Isoleucine | 106 (88-130) | 239 (223-319) | 610 (516-668) |
| N.3 | Pre-study | 284 (138-590) | 312 (155-459) | 289 (173-363) |
| | On liquid Valine | 272 (222-329) | 313 (237-403) | 362 (331-385) |
| | On liquid Isoleucine | 324 (173-575) | 366 (120-460) | 324 (167-575) |
| N.4 | Pre-study | 313 (237-401) | 279 (223-379) | 439 (381-515) |
| | On liquid Valine | 195 (153-231) | 199 (168-280) | 502 (391-592) |
| | On liquid Isoleucine | 217 (171-268) | 287 (253-248) | 354 (296-403) |
| N.5 | Pre-study | 214 (150-268) | 286 (232-364) | 443 (312-590) |
| | On liquid Valine | 349 (158-684) | 183 (116-276) | 301 (245-449) |
| | On liquid Isoleucine | 211 (170-284) | 342 (183-479) | 395 (327-461) |
| Mean | Pre-study | 356 (214-716) | 300 (243-312) | 426 (289-516) |
| (range) | On liquid Valine | 253 (195-349) | 246 (183-313) | 424 (301-513) |
| values | On liquid Isoleucine | 234 (106-324) | 327 (239-366) | 433 (324-610) |

*The patient was admitted to the hospital pre study with gastro-enteritis.

### Product Dosage and Administration

At baseline, all subjects took powdered Val or Ile supplements added to protein substitutes or fruit squash drinks. They were then transferred to 1) liquid Val and 2) liquid Ile for 4 weeks each. Four of 5 subjects took the liquid supplements directly from the syringe. One subject mixed the liquids with powdered protein substitute.

Tables 6a and 6b reports the baseline amounts of Val (Table 6a) and Ile (Table 6b) in powder form, the amounts provided as the starting dose as RTU, and dosage adjustments made during the study to ensure adequate blood Val and Ile levels.

TABLE 6a
Table 6a - Data for patients administered
with the Valine composition of Example 1.

| | Powder Daily | | | Liquid | |
|---|---|---|---|---|---|
| | dose pre study | Start | Final | Daily dose | % |
| Subject | (mg Val) | dose T0 | dose T1 | (mg Val) | Reduction |
| N.1 | 150 mg | 3 ml | 2 ml | 100 mg | 33% |
| | | 2 doses | 2 doses | | |
| | | (150 mg) | | | |
| N.2 | 200 mg | 4 ml | 2 ml | 100 mg | 50% |
| | | 3 doses | 2 doses | | |
| | | (200 mg) | | | |
| N.3 | 150 mg | 3 ml | 3 ml | 150 mg | 0% |
| | | 3 doses | 3 doses | | |
| | | (150 mg) | | | |
| N.4 | 150 mg | 3 ml | 1 ml | 50 mg | 67% |
| | | 2 doses | 1 dose | | |
| | | (150 mg) | | | |
| N.5 | 100 mg | 2 ml | 2 ml | 100 mg | 0% |
| | | 1 dose | 1 dose | | |
| | | (100 mg) | | | |
| Mean | 150 mg | 3 ml | 2.25 ml | 113 mg | 30% |

TABLE 6b
Table 6b - Data for patients administered with
the Isoleucine composition of Example 2.

| | Powder Daily | | | Liquid | |
|---|---|---|---|---|---|
| | dose pre-study | Start | Final | Daily dose | % |
| Subject | (mg Ile) | dose T1 | dose T2 | (mg Ile) | Reduction |
| N.1 | 200 mg | 8 ml | 6 ml | 150 mg | 25% |
| | | 2 doses | 2 doses | | |
| N.2 | 250 mg | 10 ml | 5 ml | 125 mg | 50% |
| | | 3 doses | 3 doses | | |
| N.3 | 150 mg | 6 ml | 5 ml | 125 mg | 17% |
| | | 3 doses | 3 doses | | |
| N.4 | 300 mg | 12 ml | 12 ml | 300 mg | 0% |
| | | 2 doses | 2 doses | | |
| N.4 | 100 mg | 4 mL | 4 mL | 100 mg | 0% |
| | | 1 dose | 1 dose | | |
| Mean | 200 mg | 8 ml | 6.4 ml | 160 mg | 18.4% |

All patients reported better palatability, overall acceptability, and ease of use with the RTU liquid supplements, as shown in Table 7. The ease of mixing the RTU Val and RTU Ile supplements was not applicable (N/A). Furthermore, one patient reported the taste of RTU Val and the aftertaste of both RTU Val and RTU Ile as not applicable, citing an inability to perceive any taste, either pleasant or unpleasant.

TABLE 7
Rating for old and new amino acid supplements expressed
as mean values for the 5 patients included in the study.

| | | | | New |
|---|---|---|---|---|
| | Old Valine | New Valine | Old Isoleucine | Isoleucine |
| | powder | liquid | powder | liquid |
| Appearance | 3.2 | 3.2 | 2.7 | 3 |
| Smell | 3.2 | 4 | 3.5 | 3 |
| Taste | 2.8* | 3.6 | 3.3 | 2.8 |
| Texture | 2 | 3.6 | 2 | 3 |
| Ease of mixing | 1.6 | N/A | 2 | N/A |
| Ease of taking | 3.2 | 5 | 2.7 | 3.7 |
| Aftertaste | 3* | 3.4 | 2.6* | 3.5 |
| Mean | 2.7 | 3.8 | 2.7 | 3.2 |

This study included five paediatric patients with classical MSUD who were well established on powdered Val and Ile supplements. It was found that that the RTU liquid supplements of Val and Ile were well tolerated, acceptable, and effective as an alternative to powdered formulations. Interestingly there was a reduction in plasma Leu levels following the introduction of both liquid Val and Ile; with RTU Val, Leu decreased from 356 to 253 μmol/L, and with Ile from 356 to 221 μmol/L. This is clinically relevant and beneficial.

Another relevant and consistent observation was that the required dose of Val and Ile decreased by 30% and 18% respectively although this was not statistically significant. For Val the mean initial dose was 150 mg/day decreasing to 113 mg/day after 4 weeks of study, and supplementation of Ile decreased from 200 mg/day to 160 mg/day. This suggests either improved efficacy, less product wastage or better adherence with the supplements.

Nutritional intake, both from natural protein and protein substitutes, remained stable throughout the study period, indicating that the change in supplement formulation did not impair dietary balance.

The liquid RTU Val and Ile was energy and carbohydrate free, so this did not require consideration in daily calculations. This will be particularly helpful when used as part of emergency feeds.

### Example 8

An alternative valine composition and an alternative isoleucine composition are shown below. Both compositions were produced by the process of Example 1.

| | Concentration |
|---|---|
| Raw Materials | % w/v |
| Valine | 5.0 |
| Sodium Methyl Parabens | 0.206 |
| Sodium Propyl Parabens | 0.0224 |
| Sodium Saccharin | 0.15 |
| Citric Acid Anhydrous | 0.25 |
| Disodium Hydrogen | 0.72 |
| Orthophosphate Dihydrate | |
| Natrosol 250HX | 0.4 |
| Strawberry Flavour (PHS-180152) | 0.5 |
| Purified Water Ph. Eur. | To 100 |
| Isoleucine | 2.5 |
| Sodium Methyl Parabens | 0.206 |
| Sodium Propyl Parabens | 0.0224 |
| Sodium Saccharin | 0.15 |
| Citric Acid Anhydrous | 0.25 |
| Disodium Hydrogen | 0.72 |
| Orthophosphate Dihydrate | |
| Natrosol 250HX | 0.4 |
| Strawberry Flavour (PHS-180152) | 0.5 |
| Purified Water Ph. Eur. | To 100 |

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the disclosure are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The disclosure is not restricted to the details of any foregoing embodiments.

What is claimed is:

1. A liquid formulation comprising valine or isoleucine.

2. The liquid formulation of claim 1, wherein the valine or isoleucine is present in an amount of from about 1.0 w/v % to about 10 w/v % based on a total liquid volume of the liquid formulation.

3. The liquid formulation of claim 1, wherein the formulation further comprises: a buffer, a preservative, a sweetener, a flavouring, a viscosity modifier or a combination thereof.

4. The liquid formulation of claim 3, wherein the buffer comprises: citric acid, trisodium citrate, disodium hydrogen orthophosphate, disodium hydrogen phosphate, sodium dihydrogen phosphate or combination thereof.

5. The liquid formulation of claim 4, wherein the buffer is present in a concentration of from about 0.5 to about 5% w/v or the buffer is present in a concentration of from about 0.1 to about 2% w/v based on a total liquid volume of the liquid formulation.

6. The liquid formulation of claim 3, wherein the preservative comprises methyl para hydroxybenzoate, ethyl para hydroxybenzoate, propyl para hydroxybenzoate, butyl para hydroxybenzoate, salts thereof or a combination thereof.

7. The liquid formulation of claim 6, wherein the in the preservative is present in a concentration of from about 0.05 to about 0.5% w/v based on a total liquid volume of the liquid formulation.

8. The liquid formulation of claim 3, wherein the viscosity modifier comprises hydroxyethylcellulose.

9. The liquid formulation of claim 1, wherein the liquid formulation is at a pH in the range of from 2 to 12.

10. The liquid formulation of claim 1, wherein the liquid formulation is at a pH in the range of from 5 to 7.

11. A method of treatment, the method comprising administering to a patient in need thereof a therapeutically effective amount of a liquid formulation comprising valine or isoleucine.

12. The method of treatment of claim 11, wherein the method of treatment is a method of treating a patient with maple syrup urine disease or a urea cycle disorder.

13. The method of treatment of claim 11, wherein the valine or isoleucine is present in an amount of from about 1.0 w/v % to about 10 w/v % based on a total liquid volume of the liquid formulation.

14. The method of treatment of claim 11, wherein the liquid formulation further comprises: a buffer, a preservative, a sweetener, a flavouring, a viscosity modifier or a combination thereof.

15. The method of treatment of claim 14, wherein the buffer comprises: citric acid, trisodium citrate, disodium hydrogen orthophosphate, disodium hydrogen phosphate, sodium dihydrogen phosphate or any combination thereof.

16. The method of treatment of claim 15, wherein the buffer is present in a concentration of from about 0.5 to about 5% w/v or the buffer is present in a concentration of from about 0.1 to about 2% w/v based on the total liquid volume of the formulation.

17. The method of treatment of claim 14, wherein the preservative comprises methyl para hydroxybenzoate, ethyl para hydroxybenzoate, propyl para hydroxybenzoate, butyl para hydroxybenzoate, salts thereof or a combination thereof.

18. The method of treatment of claim 17, wherein the preservative is present in a concentration of from about 0.05 to about 0.5% w/v based on the total liquid volume of the formulation.

19. The method of treatment of claim 11, wherein the method of treatment is a method of treating a patient with maple syrup urine disease, the method comprising
during a first administration, administering to the patient a first volume of a liquid formulation comprising 1.0 w/v % to about 10 w/v % valine and a first volume of a liquid formulation comprising 1.0 w/v % to about 10 w/v % isoleucine, and
during a second administration, administering to the patient a second volume of the first liquid formulation comprising valine and a second volume of the second liquid formulation comprising isoleucine, wherein the second volume is less than the first volume,
wherein, during the treatment, (i) the patient's plasma concentrations of valine and isoleucine remain within a predetermined range and (ii) the patient's plasma concentration of leucine decreased.

20. A kit comprising a liquid formulation comprising valine and a liquid formulation comprising isoleucine.

21. The kit of claim 20, wherein the valine is present in an amount of from about 1.0 w/v % to about 10 w/v % based on the total liquid volume of the liquid formulation that comprises valine and the isoleucine is present in an amount of from about 1.0 w/v % to about 10 w/v % based on the total liquid volume of the liquid formulation that comprises isoleucine.
